(19) United States
(12) Patent Application Publication
(10) Pub. No.: US 2013/0115624 A1
(43) Pub. Date: May 9, 2013

(54) Methods To Identify Modulators

(71) Applicants: Jay Patrick Slack, Cincinnati, OH (US); Jenny Ellen Evans Pennimpede, Cincinnati, OH (US)

(72) Inventors: Jay Patrick Slack, Cincinnati, OH (US); Jenny Ellen Evans Pennimpede, Cincinnati, OH (US)

(73) Assignee: Givaudan S.A., Vernier (CH)

(21) Appl. No.: 13/695,346

(22) PCT Filed: May 6, 2011
(86) PCT No.: PCT/EP11/57358

§ 371 Date: Jan. 15, 2013

Related U.S. Application Data
(60) Provisional application No. 61/332,331, filed on May 7, 2010.

(51) Int. Cl. G01N 33/50

(52) U.S. Cl. CPC G01N 33/5041
USPC 435/7.21; 435/7.32

(57) ABSTRACT

Disclosed are compounds that activate the human G-protein coupled receptor TAS2R41, and methods of using these compounds for identifying compounds that modulate the response of the TAS2R41 receptor. Compounds identified as modulators of the response of the receptor TAS2R41 may be used to decrease or mask the bitter taste of foods or drugs.

### TECHNICAL FIELD

Disclosed are compounds that activate the human G-protein coupled receptor TAS2R41, and methods of using these compounds for identifying compounds that modulate the response of the TAS2R41 receptor.

### BACKGROUND

One of the basic taste modalities that humans can recognise is bitter. It is understood that many compounds elicit bitter taste by interacting with G protein coupled receptors (hereinafter GPCRs).

About 25 different human bitter taste GPCRs have been identified from human genome sequences. One known GPCR is TAS2R41.

It would be beneficial to develop a method to identify compounds that modulate the response of TAS2R41 as such identified compounds could be used to decrease or mask the bitter taste of foods or drugs.

### DETAILED DESCRIPTION

It has now been found that TAS2R41 responds to cyclamate, an artificial sweetener that is known to have a bitter after taste.

This finding enables TAS2R41 to be used in screening methods to identify compounds that modulate its response. These modulating compounds may then be used in the food and pharmaceutical industries to customise taste, for example, to decrease or mask the bitter taste of foods or drugs.

According to a first illustrative aspect, there is provided a method for identifying compounds that modulate the response of TAS2R41 to cyclamate and/or structurally related compounds comprising:
I. contacting at least one cell, or membrane thereof, expressing the nucleic acid sequence encoding TAS2R41 or a functional equivalent thereof, with cyclamate and/or a structurally related compound, and at least one test compound, and
II. measuring the effect of the at least one test compound on the response of TAS2R41 to cyclamate and/or said structurally related compounds.

Structurally related compound to cyclamate include N-substituted sulfamic acid derivatives or alkali salts thereof. Examples of such compounds include, but are not limited to, N-bicyclo[2.2.1]hept-2-yl-sulfamic acid sodium salt; sodium cyclopropylsulfamate; (2-methylcyclohexyl)-sulfamic acid monosodium salt; sodium 1,2,3,4-tetrahydronaphthalen-1-ylsulfamate; sodium biphenyl-3-ylsulfamate; sodium o-tolylsulfamate; sodium propylsulfamate; sodium 3-methylbenzylsulfamate; N-(3,3-dimethylbutyl)-sulfamic acid potassium salt; N-2H-tetrazol-5-yl-sulfamic acid sodium salt; N-(5-methyl-3-isoxazolyl)-sulfamic acid sodium salt; N-1,2,4-thiadiazol-5-yl-sulfamic acid sodium salt; N-1H-benzimidazol-2-yl-sulfamic acid sodium salt; N-1H-1,2,4-triazol-5-yl-sulfamic acid sodium salt; (4,6-dimethyl-2-pyrimidinyl)-sulfamic acid monosodium salt; (3,3-dimethylbutyl)-sulfamic acid monosodium salt; sodium 4H-1,2,4-triazol-4-ylsulfamate; sodium thiazol-2-ylsulfamate; sodium isobutylsulfamate; sodium 2-methoxyethylsulfamate; sodium 2-morpholinoethylsulfamate; sodium 2-(piperidin-1-yl)ethylsulfamate; sodium 3-methylpyridin-2-ylsulfamate; sodium 3,4-dimethoxyphenethylsulfamate; sodium 1,3,4-thiadiazol-2-ylsulfamate; sodium biphenyl-3-ylsulfamate; sodium 3-methoxybenzylsulfamate, (2S,5R)-2-isopropyl-5-methylcyclohexylsulfamic acid; 2-methoxy-2-oxoethylsulfamic acid; (2-Hydroxy-ethyl)-sulfamic acid; cyclohexylmethyl-sulfamic acid; cyclobutyl-sulfamic acid; sodium cyclohexanemethylaminesulfamate; sodium (3-Methyl-butyl)-sulfamate; sodium (2-Methyl-butyl) sulfamate; sodium piperidin-1-ylsulfamate; sodium thietan-3-ylsulfamate; 2,6-dimethylcyclohexylsulfamic acid; cyclopropylsulfamic acid; sodium morpholinosulfamate; sodium cyclohexyl(methyl)sulfamate; sodium cycloheptyl(methyl)sulfamate; sodium isopropyl(tetrahydro-2H-thiopyran-4-yl)sulfamate; sodium ethyl(tetrahydro-2H-thiopyran-4-yl)sulfamate; sodium cyclobutyl(methyl)sulfamate; sodium 2-hydroxyethyl(tetrahydro-2H-thiopyran-4-yl)sulfamate; sodium isopropylsulfamate; sodium 5-methyltetrahydrothiophene-3-sulfonate; sodium sec-butylsulfamate; sodium 2,4,4-trimethylpentan-2-ylsulfamate; sodium 4-methyltetrahydrofuran-3-sulfonate; sodium butylsulfamate; sodium propylsulfamate; sodium isopentylsulfamate; sodium hexylsulfamate; sodium octylsulfamate; sodium pentadecylsulfamate; sodium octadecylsulfamate; sodium isobutylsulfamate; sodium 2-methylbutylsulfamate.

According to another illustrative embodiment, the method for identifying compounds that modulate the response of TAS2R41 to cyclamate and/or structurally related compounds, comprises an in vitro method.

According to another embodiment, the method for identifying compounds that modulate the response of TAS2R41 to cyclamate and/or structurally related compounds, comprises an in vivo method that is carried out using transgenic animals expressing the exogenous TAS2R41 receptor.

Functional equivalents of the nucleotide sequence encoding TAS2R41 include those nucleotide sequences that by virtue of the degeneracy of the genetic code possess a different nucleotide sequence to the TAS2R41 nucleotide sequence disclosed herein, but that encode for the same amino acid sequence with the same activity.

Functional equivalents encompass naturally occurring variants of the sequences described herein as well as synthetic nucleotide sequences. For example, those nucleotide sequences that are obtained by chemical synthesis or recombination of naturally existing DNA.

Functional equivalents may be the result of, natural or synthetic, substitutions, additions, deletions, replacements, or insertions of one or more nucleotides.

Examples of functional equivalents include those nucleic acid sequences comprising a sense mutation resulting from the substitution of at least one conserved amino acid which does not lead to an alteration in the activity of the polypeptide and thus they can be considered functionally neutral.

Other non-limiting examples of functional equivalents include fragments, orthologs, splice variants, single nucleotide polymorphisims, and allelic variants.

Such functional equivalents will have 75%, 80%, or 90% homology to the nucleotide sequences disclosed herein.

Nucleotide sequence homology may be determined by sequence identity or by hybridisation.

Sequence identity may be determined using basic local alignment search tool technology (hereinafter BLAST). BLAST technology is a heuristic search algorithm employed by the programs blastn which is available at http://www.ncbi.nlm.nih.gov.

If homology is determined by hybridisation, the nucleotide sequences should be considered substantially homologous provided that they are capable of selectively hybridizing to the TAS2R41 nucleotide sequence disclosed herein.

Hybridisation should be carried out under stringent hybridisation conditions at a temperature of 42° C. in a solution consisting of 50% formamide, 5×standard sodium citrate (hereinafter SSC), and 1% sodium dodecyl sulphate (hereinafter SDS). Washing may be carried out at 65° C. in a solution of 0.2×SSC and 0.1% SDS.

Background hybridization may occur because of other nucleotide sequences present, for example, in the cDNA or genomic DNA library being screened. Any signal that is less than 10 fold as intense as the specific interaction observed with the target DNA should be considered background. The intensity of interaction may be measured, for example, by radiolabelling the probe, e.g. with ³²P.

Certain, non-limiting, examples of functional equivalents of the nucleotide sequence of TAS2R41 are illustrated in SEQ ID Nos 3, 5, and 7.

The nucleotide sequence encoding TAS2R41, or a functional equivalent thereof may comprise a suitable 5′ untranslated region as well as a promoter to enable expression in host cells. This 5′ untranslated region may also comprise other operators or motifs that influence the efficiency of transcription or translation, and/or tags.

The nucleotide sequence encoding the TAS2R41 receptor may also comprise a suitable 3′ untranslated region as well as a stop codon, this 3′ untranslated region may also comprise other signals such as a signal for transcriptional termination.

Non limiting examples of operators or motifs that influence transcription or translation include, but are not limited to, signals required for efficient polydenylation of the transcript, ribosome binding sites, recognition sites e.g. EcoR1.

Non limiting examples of tags include, but are not limited to, membrane export tags and tags used for detection of TAS2R41 including, but not limited to, immuno detection tags.

Any of the known membrane export tags or tags used for detection of proteins may be used.

Non limiting examples of membrane export tags include, but are not limited to, tags from somatostatin such as rat somatostatin (STT, SEQ ID NO:3), rhodopsin or bovine tag/fragments, such as the 39 N-terminal amino acid of rhodopsin or bovine rhodopsin (see for example in Krautwurst et al. 1998, Cell 95(7):917-26), or the relevant fragment from another membrane protein, for example, without limitation, about 7 to about 100 N-terminal aminoacids of a membrane protein.

Any of the known tags used for detection of GPCRs may be used. Non limiting examples of such tags are immuno detection tags. Non limiting examples of immuno detection tags include FLAG® tags (Sigma) with the aminoacid sequence [(M)DYKDDDDK)], HA tags [YPYDVPDYA], c-MYC tags [EQKLISEEDL], HIS tags [HHHHHH], HSV tags [QPELAPEDPED], VSV-G tags [YTDIEMNRLGK], V5 tags [GKPIPNPLLGLDST].

It is well within the purview of the person skilled in the art to decide upon suitable tags, and operators or motifs that influence transcription or translation, depending on the host cells in question and the desired result.

According to an illustrative embodiment, the nucleotide sequence encoding the TAS2R41 receptor, or a functional equivalent thereof, comprises a HSV tag and a rat somatostatin tag (SST).

Suitable cells for use in the methods disclosed herein include prokaryote and eucaryotic cells, non limiting examples of which include, bacteria cells, mammalian cells, yeast cells, or insect cells (including Sf9), amphibian cells (including melanophore cells), or worm cells including cells of Caenorhabditis (including Caenorhabditis elegans).

According to other embodiments, the cell used in the method for identifying modulators of the TAS2R41 receptor comprises a mammalian cell.

Non limiting examples of suitable mammalian cells include, COS cells (including Cos-1 and Cos-7), CHO cells, HeLa cells, HEK293 cells, HEK293T cells, HEK293 T-Rex™ cells, other transfectable eucaryotic cell lines, and the like.

According to certain illustrative embodiments, the cell comprises a mammalian cell selected from CHO, COS, HeLa and Hek-293.

For use in the aforementioned method cells may be isolated cells or alternatively they may be components of tissue including, but not limited to, mammalian tissue and transgenic animal tissue.

The cells used in the method may naturally express a nucleotide sequence encoding TAS2R41, or a functional equivalent thereof, or they may be recombinant cells expressing a nucleotide sequence encoding TAS2R41, or a functional equivalent thereof.

Recombinant cells may be transfected with a nucleotide sequence or an amino acid sequence encoding TAS2R41, or a functional equivalent thereof, transiently or stably, as is well known in the art.

Isolation and expression of TAS2R41, or functional equivalents thereof, may be effected by well established cloning techniques using probes or primers constructed based on the nucleic acid sequence disclosed herein. Once isolated, the nucleotide sequences may be amplified through the polymer chain reaction (hereinafter PCR).

Any known method for introducing nucleotide sequences into host cells may be used. It is only necessary that the particular genetic engineering procedure used be capable of successfully introducing the relevant genes into the host cell capable of expressing the proteins of interest. These methods may involve introducing cloned genomic DNA, cDNA, synthetic DNA or other foreign genetic material into a host cell and include the use of calcium phosphate transfection, polybrene, protoplast fusion, electroporation, liposomes, microinjection, expression vectors, and the like.

According to other embodiments, expression vectors may be used to infect or transfect host cells with the nucleic acid sequence encoding TAS2R41, or a functional equivalent thereof, for use in the aforementioned method.

Expression vectors, both as individual expression vectors or as libraries of expression vectors, comprising at least one nucleic acid sequences encoding TAS2R41 and/or functional equivalents thereof, may be introduced and expressed in a cell's genome, a cell's cytoplasm, or a cell's nucleus by a variety of conventional techniques.

It is well within the purview of the person skilled in the art to decide upon a suitable technique.

Any suitable expression vector may be used. Non limiting examples of types of vectors include bacteriophage, plasmid, or cosmid DNA expression vectors, yeast expression vectors; viral expression vectors (for example baculovirus), or bacterial expression vectors (for example pBR322 plasmids).

More specific non limiting examples include, plasmids including pBR322-based plasmids, pSKF, and pET23D, and fusion expression systems, for example, GST and LacZ, SV40 vectors, cytomegalovirus vectors, papilloma virus vectors, and vectors derived from Epstein-Barr virus, pMSG, pAV009/A⁺, pMTO10/A⁺, pMAMneo-5, baculovirus pDSVE, pcDNA3.1, pIRES.

Further examples of vectors that may be used are described in “G-protein coupled receptors (Signal Transduction Series)”; Editors: Tatsuya Naga and Gabriel Berstein, 1st ed., CRC Press—Boca Raton Fla.; September 1999.

It is well within the purview of the person skilled in the art to decide upon a suitable expression vector depending on the host cells in question and the desired effect.

According to an illustrative embodiment, the expression vector may be selected from pcDNA3.1Zeo or pcDNA5/FRT (Invitrogen, Carlsbad, Calif., US).

After transfection, the transfected cells may be cultured using standard culturing conditions well known in the art. It will be apparent to the skilled person that different cells require different culture conditions including appropriate temperature and cell culture media. It is well within the purview of the person skilled in the art to decide upon culture conditions depending on the cells in question and the desired end result.

Information on appropriate culturing media and conditions with respect to certain cells may be found on the American type culture collection (ATCC) Website: http://www.lqcstandards-atcc.orq/Home/tabid/477/Defaultaspx

In a particular illustrative embodiment the cells used were Hek-293 cells, the culture medium was Dulbecco's modified Eagle's medium (DMEM) with 10% (v/v) heat-inactivated fetal bovine serum. Cells were incubated overnight at 37° C.

TAS2R41 may be overexpressed by placing it under the control of a strong constitutive promoter, for example, the CMV early promoter. Alternatively, certain mutations of conserved GPCR amino acids or amino acid domains can be introduced to render the employed TAS2R41 constitutively active.

The effect of a test compound on the response of TAS2R41 may be determined by comparing the response of TAS2R41 to cyclamate and/or structurally related compounds in both the absence and presence of the test compound.

The method for identifying compounds that modulate the response of TAS2R41 to cyclamate and/or structurally related compounds may comprise:
I. contacting at least one cell, or membrane thereof, expressing the nucleic acid sequence encoding TAS2R41 or a functional equivalent thereof, with cyclamate and/or structurally related compounds
II. measuring the response of TAS2R41 to the cyclamate and/or structurally related compounds
III. contacting at least one cell, or membrane thereof, with at least one test compound, and cyclamate and/or structurally related compounds
IV. measuring the response of TAS2R41 to the cyclamate and/or structurally related compounds in the presence of the test compound
V. calculating the change in the response of TAS2R41 to the cyclamate and/or structurally in the presence of the test compound.

The response of TAS2R41 may be determined by measuring the change in any parameter that is directly or indirectly under the influence of TAS2R41. These parameters include physical, functional, and chemical effects.

Examples of measurable parameters include, but are not limited to, changes in ion flux, membrane potential, current flow, transcription, G-protein binding, GPCR phosphorylation or dephosphorylation, signal transduction, receptor-ligand interactions, intracellular messenger concentrations e.g. phospholipase C, adenylate cyclase, guanylate cyclase, phospholipase, cAMP, cGMP, IP3, DAG, intracellular Ce^{2+,} ligand binding, neurotransmitter levels, GTP-binding, GTPase, adenylate cyclase, phospholipid-breakdown, diacylglycerol, inositol triphosphate, arachidonic acid release, protein kinase c (PKC), MAP kinase tyrosine kinase, and ERK kinase.

The aforementioned parameters may be measured by any means known to those skilled in the art, for example, changes in the spectroscopic characteristics e.g. fluorescence, absorbance, refractive index), hydrodynamic (e.g. shape), chromatographic, or solubility properties, patch clamping, voltage-sensitive dyes, whole cell currents, radioisotope efflux, inducible markers, oocyte TAS2R41 gene expression, tissue culture TAS2R41 cell expression, transcriptional activation of TAS2R41 genes, ligand binding assays, voltage, membrane potential and conduction changes; ion flux assays, assays that measure changes in parameters of the transduction pathways such as intracellular IP₃ and Ca²⁺, diacylglycerol/DAG, arachinoid acid, MAP kinase or tyrosine kinase, assays based on GTP-binding, GTPase, adenylate cyclase, phospholipid-breakdown, diacylglycerol, inositol triphosphate, arachidonic acid release, PKC, kinase and transcriptional reporters, or by other G-protein specific assays such as labeling with GTPγS.

Various suitable assays are described in WO 01/18050, US20050032158, paragraphs [0169] to [0198], which is incorporated herein by reference, and hereinbelow.

It is well within the purview of the person skilled in the art to decide on a suitable measurement technique.

According to certain embodiments, the effect of test compounds on the response of TAS2R41 to cyclamate and/or structurally related compounds is determined by measuring the change in concentration of the intracellular messenger IP3 and/or ca²⁺

To enable the measurement of certain parameters it may be necessary or desirable to link a G-protein or a reporter gene to TAS2R41.

Any suitable G-protein or reporter gene may be used and it is well within the purview of the person skilled in the art to decide upon an appropriate G-protein or reporter gene depending on the desired response.

Examples of reporter genes include, but are not limited to: luciferase, CAT, GFP, β-lactamase, β-galactosidase, and the so-called “immediate early” genes, c-fos proto-oncogene, transcription factor CREB, vasoactive intestinal peptide (VIP) gene, the somatostatin gene, the proenkephalin gene, the phosphoenolpyruvate carboxy-kinase (PEPCK) gene, genes responsive to NF-κB, and AP-1-responsive genes (including the genes for Fos and Jun, Fos-related antigens (Fra) 1 and 2, IκBα, ornithine decarboxylase, and annexins I and II).

In general, reporter genes are linked to one or more transcriptional control elements or sequences necessary for receptor-mediated regulation of gene expression, including but not limited to, one or more promoter, enhancer and transcription-factor binding site necessary for receptor-regulated expression.

It is well within the purview of the person skilled in the art to decide on appropriate transcriptional control elements or sequences depending on the effect desired.

Examples of G-proteins include, but are not limited to, chimeric G-proteins based on Gaq-Gustducin as described in WO 2004/055048, in particular Gα16 or Gals.

According to certain embodiments, a G-protein is linked to TAS2R41.

The G-protein may be the chimeric G-protein G alpha 16-gustducin 44 (also known as “G16gust44” as used herein) which provides for enhanced coupling to taste GPCRs. This G-protein is described in detail in WO 2004/055048, which is incorporated herein by reference.

Compounds that modulate the response of TAS2R41 to cyclamate and/or structurally related compounds (hereinafter modulators) may be categorized as one or more of the following: agonist, antagonist, inhibitor or enhancer.

The term agonist as used herein is used to describe a compound which activates TAS2R41 and brings about an intracellular response. Cyclamate is an agonist of TAS2R41.

The term antagonist as used herein is used to describe a compound which does not activate TAS2R41, and consequently does not bring about an intracellular response, but that binds to TAS2R41 at the same (competitive antagonist) or at a different site (allosteric antagonist) as an agonist such as cyclamate and or structurally related compounds. Compounds that are antagonists thereby prevent or dampen the intracellular response mediated by the interaction of agonists such as cyclamate and/or structurally related compounds, with TAS2R41.

The term inhibitor as used herein is used to describe a compound that prevents or decreases receptor activation mediated by the interaction of agonists, such as cyclamate and/or structurally related compounds, with TAS2R41.

The term enhancer as used herein is used to describe a compound that increases the receptor activation mediated through the interaction of agonists, such as cyclamate and/or structurally related compounds, with TAS2R41. Compounds that are enhancers thereby cause an increase in the intracellular response mediated by agonists such as cyclamate and/or structurally related compounds.

Modulators may be categorized as one or more of the aforementioned terms, for example, a compound may act as an enhancer in a certain concentration range, but act as an inhibitor in another concentration range. For this reason, compounds may be tested at different concentrations.

Various types of compounds may be modulators, non limiting examples of the various types of compounds include small molecules, peptides, proteins, nucleic acids, antibodies or fragments thereof. These compounds may be derived from various sources including synthetic or natural, extracts of natural material, for example from animal, mammalian, insect, plant, bacterial or fungal cell material or cultured cells, or conditioned medium of such cells.

The method described herein may be used to screen libraries for modulators.

The assays may be run in high throughput screening methods that involve providing a combinatorial chemical or peptide library containing a large number of potential modulators. Such libraries may be screened in one or more of the assays described herein to identify those library compounds (particular chemical species or subclasses) that have an effect on the response of TAS2R41 to cyclamate and/or structurally related compounds.

The modulators thus identified can then be directly used or may serve as leads to identify further modulators by making and testing derivatives.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

A combinatorial chemical library is available from Aldrich (Milwaukee, Wis.).

Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.).

Libraries of natural compounds in the form of bacterial, fungal, plant and animal extracts are commercially available for example from Pan Laboratories (Bothell, Wash.) or MycoSearch (NC), or are readily producible by methods well known in the art. Additionally, natural and synthetically produced libraries and compounds are readily modified through conventional chemical, physical, and biochemical means.

Other libraries which may be used include protein/expression libraries, cDNA libraries from natural sources, including, for example, foods, plants, animals, bacteria, libraries expressing randomly or systematically mutated variants of one or more polypeptides, genomic libraries in viral vectors that are used to express the mRNA content of one cell or tissue.

A modulator identified by a method described herein may easily be tested by simple sensory experiments using a panel of flavorists or test persons. The identified modulator may be tasted in water together with cyclamate and/or structurally related compounds, and compared to a negative control just containing cyclamate and/or structurally related compounds in water without the modulator.

In another aspect, there is provided a kit, for example a screening kit or high throughput screening kit, for identifying compounds that modulate the response of TAS2R41 to cyclamate and/or structurally related compounds comprising:
I. at least one recombinant cell expressing the nucleotide sequence encoding TAS2R41, or a functional equivalent thereof, and
II. cyclamate and/or a structurally related compound.

The kit may be used to carry out the method, as herein disclosed, for identifying compounds that modulate the response of TAS2R41 to cyclamate and/or structurally related compounds.

Cyclamate and/or a structurally related compound may be provided in a concentration of 0.01 mM to 500 mM, 0.1 mM to 200 mM, or 0.01 mM to 100 mM.

As detailed above recombinant cells expressing TAS2R41 may additionally express reporter genes, G-proteins, tags, and operators and motifs that influence the efficiency of transcription or translation.

In certain embodiments, the recombinant cells additionally express a G-protein.

In another embodiment, the G-protein is the chimeric G-protein G16gust44.

The aforementioned kit may also include optional components such as; a suitable medium for culturing the provided recombinant cells, and a solid support to grow the cells on, for example, a cell culture dish or microtiter plate. The optional components will be readily available to the skilled person.

In another aspect, there is provided a method of using the aforementioned kit to identify compounds that modulate the response of TAS2R41 to cyclamate and/or structurally related compounds comprising:
I. growing at least one recombinant cell expressing the nucleotide sequence encoding TAS2R41, or a functional equivalent thereof, on a solid support in a culture medium,
II. adding one or more test compound and cyclamate and/or structurally related compounds to the culture medium, and
III. measuring the effect of the test compound on the response of TAS2R41 to cyclamate and/or structurally related compounds.

As stated hereinabove, the effect of a test compound on the response of TAS2R41 may be determined by comparing the response of TAS2R41 to cyclamate and/or structurally related compounds in the absence and presence of the test compound.

In an illustrative embodiment, the method of using the aforementioned kit to identify compounds that modulate the response of TAS2R41 to cyclamate and/or structurally related compounds comprises:
I. growing at least one recombinant cell expressing the nucleotide sequence encoding TAS2R41, or a functional equivalent thereof, on a solid support in a culture medium
II. adding cyclamate and/or structurally related compounds to the culture medium
III. measuring the response of TAS2R41 to the cyclamate and/or structurally related compounds
IV. growing at least one recombinant cell expressing the nucleotide sequence encoding TAS2R41, or a functional equivalent thereof, on a solid support in a culture medium
V. adding at least one test compound, and cyclamate and/or structurally related compounds to the culture medium
VI. measuring the response of TAS2R41 to the cyclamate and/or structurally related compounds in the presence of the test compound
VII. calculating the change in the response of TAS2R41 to cyclamate and/or structurally related compounds in the presence of the test compound.

The test compounds should be added to the culture medium at concentrations from about 0.01 mM to 500 mm, 0.1 mM to 200 mM, or 0.01 mM to 100 mM.

Cyclamate and/or structurally related compounds should be added to the culture medium in a concentration from 0.01 mM to 500 mM, 0.1 mM to 200 mM, or 0.01 mM to 100 mM.

In an illustrative embodiment 100m of cyclamate is added to the culture medium

As mentioned herein, it is possible to measure a variety of parameters to determine the effect of a test compound on the response to TAS2R41, or a functional equivalent thereof, to cyclamate and/or structurally related compounds. Some of these are now detailed in greater specificity.

Throughout these descriptions the term “receptor(s)” refers to the TAS2R41 receptor and the term “known agonist(s)” refers to cyclamate and/or structurally related compounds.

### Detection of Changes of Cytoplasmic Ions or Membrane Voltage:

Cells are loaded with ion sensitive dyes to report receptor activity, as described in detail in “G-protein coupled receptors (Signal Transduction Series)”, CRC Press 1999; 1st Edition; Eds Haga and Berstein. Changes in the concentration of ions in the cytoplasm or membrane voltage are measured using an ion sensitive or membrane voltage fluorescent indicator, respectively.

### Calcium Flux:

Intracellular calcium release induced by the activation of receptors is detected using cell-permeant dyes that bind to calcium. The calcium-bound dyes generate a fluorescence signal the strength of which is proportional to the rise in intracellular calcium. The methods allows for rapid and quantitative measurement of receptor activity.

Cells used are transfected cells that co-express the receptor and a G-protein which allows for coupling to the phospholipase C pathway. Negative controls include cells or their membranes not expressing the receptor (mock transfected), to exclude possible non-specific effects of the test compound.

The calcium flux detection protocol is described in detail in “G-protein coupled receptors (Signal Transduction Series)”; Editors: Tatsuya Naga and Gabriel Berstein, 1st ed., 424 pp.CRC Press—Boca Raton Fla.; September 1999. An adapted version with is summarised below:

Day 0: 96-well plates are seeded with 8.5K cells per well and maintained at 37° C. overnight in nutritive growth media.
Day 1: Cells are transfected using 150 ng of receptor DNA and 0.3 μl of Lipofectamine 2000 (Invitrogen) per well. Transfected cells are maintained at 37° C. overnight in nutritive growth media.
Day 2: Growth media is discarded and cells are incubated for 1 hour (at 37° C. in the dark) with 50 μl of calcium assay solution consisting of 1.5 μM Fluo-4 AM (Molecular Probes) and 2.5 mM probenicid dissolved in C1 buffer solution which contains 130 mM NaCl, 5 mM KCl, 10 mM Hepes, 2 mM CaCl2 and 10 mM glucose (pH 7.4) at 37° C. 125 μl of C1 buffer is added to each well and the plate is further incubated for 30 minutes at room temperature in the dark.
Buffer solutions are discarded and the plate is washed 5 times with 100 μl C1 buffer as a washing buffer and cells are reconstituted in 200 μl of C1 buffer.

Then the plate is placed in a fluorescent microplate reader, for example, the Flexstation (Molecular Devices) or the FLIPR (Molecular Devices) and receptor activation is initiated following addition of 20 μl of a known concentration agonist stock solution. Fluorescence is continuously monitored for 15 seconds prior to known agonist addition and for 45-110 seconds after known agonist addition.

### Receptor Activation Levels May be Defined as Follows:

By % Activation=(Maximum fluorescence−baseline fluorescence/baseline fluorescence)*100 or Fluorescence Increase=Maximum Fluorescence−baseline fluorescence, where baseline fluorescence represents the average fluorescence levels prior to known agonist addition.

By an increase in peak fluorescence (F) which is normalized to the baseline fluorescence (F0) using the equation ΔF/F=(F−F0)/F0 in which F is the peak fluorescence signal and F0 is the baseline fluorescence signal (baseline fluorescence represents the mean fluorescence calculated for the first 10 to 15 seconds prior to ligand addition).

By Peak Fluorescence Increase=Maximum Fluorescence−Baseline Fluorescence in which Baseline Fluorescence represents the average fluorescence level prior to known agonist addition.

The identification of a compound that modulated the response of the receptor to a known agonist is performed as described above subject to the following modifications. The signals are compared to the baseline level of receptor activity obtained from recombinant cells expressing the receptor in the presence of agonist but in the absence of a test compound. An increase or decrease in receptor activity, for example of at least 2 fold, at least 5 fold, at least 10 fold, at least a 100 fold, or more identifies a compound that modulates the response of the receptor to a known agonist.

Alternatively, the identification involves an increase or decrease in fluorescence intensity of, for example, 10% or more, when compared to a sample without a compound that modulates the response of the receptor, or when compared to a sample with a compound that modulates the response of the receptor but in cells that do not express the receptor (mock-transfected cells).

### Adenylate Cyclase Activity:

Assays for adenylate cyclase activity are performed, for example, as described in detail by Kenimer & Nirenberg, 1981, Mol. Pharmacol. 20: 585-591. Reaction mixtures are incubated usually at 37° C. for less than 10 minutes. Following incubation, reaction mixtures are deproteinized by the addition of 0.9 ml of cold 6% trichloroacetic acid. Tubes are centrifuged and each supernatant solution is added to a Dowex AG50W-X4 column. The cAMP fraction from the column is eluted with 4 ml of 0.1 mM imidazole-HCl (pH 7.5) into a counting vial in order to measure the levels of cAMP generated following receptor activation by a known agonist. Control reactions should also be performed using protein homogenate from cells that do not express the receptor.

### IP3/Ca²⁺ Signals:

In cells expressing G-proteins, signals corresponding to inositol triphosphate (IP3)/Ca²⁺ and thereby receptor activity can be detected using fluorescence. Cells expressing a receptor may exhibit increased cytoplasmic calcium levels as a result of contribution from both intracellular stores and via activation of ion channels, in which case it may be desirable, although not necessary, to conduct such assays in calcium-free buffer, optionally supplemented with a chelating compounds such as EDTA, to distinguish fluorescence response resulting from calcium release from internal stores.

Phospholipase C/intracellular Ca²⁺ signals:

A receptor is expressed in a cell with a G-protein that links the receptor to a phospholipase C signal transduction pathway. Changes in intracellular Ca²⁺ concentration are measured, for example using fluorescent Ca²⁺ indicator dyes and/or fluorometric imaging.

### GTPase/GTP Binding:

For a receptor, a measure of receptor activity is the binding of GTP by cell membranes containing the receptor. Measured is the G-protein coupling to membranes by detecting the binding of labelled GTP. Membranes isolated from cells expressing the receptor are incubated in a buffer containing 35S-GTPγS and unlabelled GDP. Active GTPase releases the label as inorganic phosphate, which is detected by separation of free inorganic phosphate in a 5% suspension of activated charcoal in 20 mM H₃PO₄, followed by scintillation counting. The mixture is incubated and unbound labelled GTP is removed by filtration onto GF/B filters. Bound and labelled GTP is measured by liquid scintillation counting. Controls include assays using membranes isolated from cells not expressing a receptor (mock-transfected), in order to exclude possible non-specific effects of the test compound. The method is described in detail by Traynor and Nahorski, 1995, Mol. Pharmacol. 47: 848-854.

To identify compounds that modulate the response of a receptor to a known agonist, as described herein, a change (increase or decrease) of 10% or more in GTP binding or GTPase activity is usually sufficient. However, to identify compounds, other than known agonists that are themselves agonists, the assays described hereinabove are performed subject to the following modifications. A compound is identified as an agonist usually if the activity is at least 50% of that of a known agonist when the compound is present at 100 mM or less, for example 10 to 500 μM, for example about 100 μM, or if it will induce a level the same as or higher than that induced by a known agonist.

### Microphysiometer or Biosensor:

Such assays can be performed as described in detail in Hafner, 2000, Biosens. Bioelectron. 15: 149-158.

### Arachinoid Acid:

The intracellular level of arachinoid acid is employed as an indicator of receptor activity. Such a method is described in detail by Gijon et al., 2000,J. Biol. Chem., 275:20146-20156.

cAMP/cGMP:

Intracellular or extracellular cAMP is measured using a cAMP radioimmunoassay (RIA) or cAMP binding protein, for example as described by Horton & Baxendale, 1995, Methods Mol. Biol. 41: 91-105. Alternatively, a number of kits for the measurement of cAMP are commercially available, for example the High Efficiency Fluorescence Polarization-based homogeneous assay by LJL Biosystems and NEN Life Science Products. Alternatively, the intracellular or extracellular levels of cGMP may measured using an immunoassay. For example, the method described in Felley-Bosco et al., Am. J. Resp. Cell and Mol. Biol., 11:159-164 (1994), may be used to determine the level of cGMP. Alternatively an assay kit for measuring cAMP and/or cGMP as described in U.S. Pat. No. 4,115,538 can be used.

Negative controls with mock-transfected cells or extracts thereof to exclude possible non-specific effects of test compounds may be used.

### DAG/IP3:

Second messengers Diacylglycerol (DAG) and/or inositol triphosphate (IP3), which are released by Phospholipid breakdown, that is caused by receptor activity, can be detected and used as an indicator of receptor activity, for example as described in Phospholipid Signalling Protocols, edited by Ian M. Bird, Totowa, N.J., Humana Press, 1998. Alternatively, kits for the measurement of inositol triphosphates are available commercially from Perkin Elmer and CisBio International.

Negative controls with mock-transfected cells or extracts thereof to exclude possible non-specific effects of test compounds may be used.

### PKC Activity:

Growth factor receptor tyrosine kinases can signal via a pathway involving activation of Protein Kinase C(PKC), which is a family of phospholipid- and calcium-activated protein kinases.

Increases in gene products induced by PKC show PKC activation and thereby receptor activity. These gene products include, for example, proto-oncogene transcription factor-encoding genes (including c-fos, c-myc and c-jun), proteases, protease inhibitors (including collagenase type I and plasminogen activator inhibitor), and adhesion molecules (including intracellular adhesion molecule I (ICAM I)).

PKC activity may be directly measured as described by Kikkawa et al., 1982, J. Biol. Chem. 257: 13341, where the phosphorylation of a PKC substrate peptide, which is subsequently separated by binding to phosphocellulose paper, is measured. It can be used to measure activity of purified kinase, or in crude cellular extracts. Protein kinase C sample can be diluted in 20 mM HEPES/2 mM DTT immediately prior to the assay.

An alternative assay can be performed using the Protein Kinase C Assay Kit commercially available by PanVera.

The above-described PKC assays may be performed on extracts from cells expressing a receptor. Alternatively, activity may be measured through the use of reporter gene constructs driven by the control sequences of genes activated by PKC activation.

Negative controls with mock-transfected cells or extracts thereof to exclude possible non-specific effects of test compounds may be used.

### MAP Kinase Activity:

MAP kinase activity can be measured using commercially available kits, for example, the p38 MAP Kinase assay kit by New England Biolabs, or the FlashPlate™ MAP Kinase assays by Perkin-Elmer Life Sciences. p42/44 MAP kinases or ERK1/2 can be measured to show GPCR (TAS2R41) activity when cells with Gq and Gi coupled GPCRs are used, and an ERK1/2 assay kit is commercially available by TGR Biosciences, which measures the phosphorylation of endogenous ERK1/2 kinases following GPCR activation.

Alternatively, direct measurements of tyrosine kinase activity through known synthetic or natural tyrosine kinase substrates and labelled phosphate are well known; the activity of other types of kinases (for example, Serine/Threonine kinases) can be measured similarly.

All kinase assays can be performed with both purified kinases and crude extracts prepared from cells expressing one or more receptor.

The substrates of kinases that are used can be either full-length protein or synthetic peptides representing the substrate. Pinna & Ruzzene (1996, Biochem. Biophys. Acta 1314: 191-225) lists a number of phosphorylation substrate sites useful for detecting kinase activities. A number of kinase substrate peptides are commercially available. One that is particularly useful is the “Src-related peptide,” RRLIEDAEYAARG (commercially available from Sigma), which is a substrate for many receptor and nonreceptor tyrosine kinases. Some methods require the binding of peptide substrates to filters, then the peptide substrates should have a net positive charge to facilitate binding. Generally, peptide substrates should have at least 2 basic residues and a free-amino terminus. Reactions generally use a peptide concentration of 0.7-1.5 mM.

Negative controls with mock-transfected cells or extracts thereof to exclude possible non-specific effects of test compounds may be used.

### Transcriptional Reporters/TAS2R41-Responsive Promoter/Reporter Gene:

To identify compounds that modulate the response of the receptor to known agonists with reporter gene assays, an at least 2-fold increase or 10% decrease in the signal is significant. A known agonist stimulates for example at least 2-fold, 5-fold, 10-fold or more when comparing activity in presence and absence of the test compound.

The intracellular signal initiated by binding of a known agonist to a receptor sets in motion a cascade of intracellular events, the ultimate consequence of which is a rapid and detectable change in the transcription or translation of one or more genes.

The activity of the receptor can therefore be determined by measuring the expression of a reporter gene driven by a transcriptional control element or sequence i.e a promoter responsive to receptor activation.

Controls for transcription assays include both cells not expressing a receptor, but carrying the reporter gene construct, and cells expressing a receptor and the reporter gene but not expressing a transcriptional control elements or sequences i.e promoter construct.

Compounds that modulate the response of the receptor to known agonists as shown by reporter gene activation can be verified by using other transcriptional control elements or sequences i.e. promoters and/or other receptors to verify receptor specificity of the signal and determine the spectrum of their activity, thereby excluding any non-specific signals, for example non-specific signals via the reporter gene pathway.

### Inositol Phosphates (IP) Measurement:

Phosphatidyl inositol (P1) hydrolysis may be determined as described in U.S. Pat. No. 5,436,128, which involves labelling of cells with 3H-myoinositol for at least 48 hours or more. The labelled cells are contacted with a test compound for one hour, then these cells are lysed and extracted in chloroform-methanol-water. This is followed by separating the inositol phosphates by ion exchange chromatography and quantifying them by scintillation counting. For known agonists, fold stimulation is determined by calculating the ratio of counts per minute (cpm) in the presence of a test compound, to cpm in the presence of buffer control. Likewise, for inhibitors and antagonists, fold inhibition is determined by calculating the ratio of cpm in the presence of test compound, to cpm in the presence of buffer control (which may or may not contain agonist).

### Binding Assays:

Binding assays are well known in the art and can be tested in solution, in a bilayer membrane, optionally attached to a solid phase, in a lipid monolayer, or in vesicles. Binding of a modulator to a receptor can be determined, for example, by measuring changes in spectroscopic characteristics (for example fluorescence, absorbance, or refractive index), hydrodynamic methods (employing for example shape), chromatography, measuring solubility properties of a receptor. In one embodiment, binding assays are biochemical and use membrane extracts from cells/tissue expressing recombinant receptors. A binding assay may, for example, be performed as described for T1 Rs by Adler et al. in US20050032158, paragraphs [0169] to [0198].

Compounds structurally related to cyclamate have been referred to throughout this text, examples of such compounds include, but are not limited to, sulfamic acid, N-bicyclo[2.2.1]hept-2-yl-, sodium salt sodium cyclopropylsulfamate; sulfamic acid, (2-methylcyclohexyl)-, monosodium salt; sodium 1,2,3,4-tetrahydronaphthalen-1-ylsulfamate; sodium biphenyl-3-ylsulfamate; sodium o-tolylsulfamate; sodium propylsulfamate; sodium 3-methylbenzylsulfamate; sulfamic acid, N-(3,3-dimethylbutyl)-, potassium salt; sulfamic acid, N-2H-tetrazol-5-yl-, sodium salt; sulfamic acid, N-(5-methyl-3-isoxazolyl)-, sodium salt; sulfamic acid, N-1,2,4-thiadiazol-5-yl-, sodium salt; sulfamic acid, N-1H-benzimidazol-2-yl-, sodium salt; sulfamic acid, N-1H-1,2,4-triazol-5-yl-, sodium salt; sulfamic acid, (4,6-dimethyl-2-pyrimidinyl)-, monosodium salt; sulfamic acid, (3,3-dimethylbutyl)-, monosodium salt; sodium 4H-1,2,4-triazol-4-ylsulfamate; sodium thiazol-2-ylsulfamate; sodium isobutylsulfamate; sodium 2-methoxyethylsulfamate; sodium 2-morpholinoethylsulfamate; sodium 2-(piperidin-1-yl)ethylsulfamate sodium 3-methylpyridin-2-ylsulfamate; sodium 3,4-dimethoxyphenethylsulfamate; sodium 1,3,4-thiadiazol-2-ylsulfamate; sodium biphenyl-3-ylsulfamate; sodium 3-methoxybenzylsulfamate, (2S,5R)-2-isopropyl-5-methylcyclohexylsulfamic acid; 2-methoxy-2-oxoethylsulfamic acid; (2-Hydroxy-ethyl)-sulfamic acid; cyclohexylmethyl-sulfamic acid; cyclobutyl-sulfamic acid; sodium N-cyclopropylsulfamate; sodium cyclohexanemethylaminesulfamate; sodium (3-Methyl-butyl)-sulfamate; sodium (2-Methyl-butyl) sulfamate; sodium piperidin-1-ylsulfamate sodium thietan-3-ylsulfamate; 2,6-dimethylcyclohexylsulfamic acid; cyclopropylsulfamic acid sodium morpholinosulfamate; sodium cyclohexyl(methyl)sulfamate; sodium cycloheptyl(methyl)sulfamate; sodium isopropyl(tetrahydro-2H-thiopyran-4-yl)sulfamate; sodium ethyl(tetrahydro-2H-thiopyran-4-yl)sulfamate; sodium cyclobutyl(methyl)sulfamate; sodium azepane-1-sulfonate; sodium azocane-1-sulfonate; sodium azonane-1-sulfonate; sodium pyrrolidine-1-sulfonate sodium 2-hydroxyethyl(tetrahydro-2H-thiopyran-4-yl)sulfamate; sodium 2-methyltetrahydrothiophene-3-sulfonate; sodium 4-methyltetrahydrothiophene-3-sulfonate; sodium isopropylsulfamate; sodium 5-methyltetrahydrothiophene-3-sulfonate; sodium sec-butylsulfamate; sodium 2,4,4-trimethylpentan-2-ylsulfamate; sodium 4-methyltetrahydrofuran-3-sulfonate; sodium butylsulfamate; sodium propylsulfamate; sodium isopentylsulfamate; sodium hexylsulfamate; sodium octylsulfamate; sodium pentadecylsulfamate; sodium octadecylsulfamate; sodium isobutylsulfamate; sodium 2-methylbutylsulfamate.

### SEQUENCE LISTINGS

SEQ ID No. 1,—Nucleic acid sequence encoding the TAS2R41 receptor.
SEQ ID Nos. 3, 5, 7—Nucleic acid sequences encoding functional equivalents of the TAS2R41 receptor.
SEQ ID No. 2—Amino acid sequence of the TAS2R41 receptor.
SEQ ID Nos. 4, 6, 8—Amino acid sequences of functional equivalents of the TAS2R41 receptor.
SEQ ID No. 9—Nucleic acid sequence encoding an SST tag.
SEQ ID No. 10—Amino acid sequence of SST tag.
SEQ ID No. 11—Nucleic acid sequence encoding an HSV tag. This sequence includes a thymine nucleoside to get into frame, a NotI site and a stop codon.
SEQ ID No. 12—Amino acid sequence of the HSV tag.

The nucleic acid sequence, and corresponding amino acid sequence encoding TAS2R41 referred to hereinabove, are known and have been published by The National Center for Biotechnology Information (NCBI) under the following reference sequence (RefSeq) numbers:

Nucleotide sequence: NM_{—}176883.2 GI: 259287
Amino acid sequence: AAM_{—}19323.1 GI: 20336521

There now follows a series of examples that serve to illustrate the above-described methods. The following examples are merely illustrative and should not be construed as limiting the described subject matter including the methods and kit in any manner.

### EXAMPLES

All examples use DNA sequences based on the mRNA for the human bitter taste receptor TAS2R41 disclosed herein.

### Example 1

Generation of Human TAS2R41 Expression Vector

The full length gene of human TAS2R41 (SEQ ID NO:1) was amplified by polymerase chain reaction (PCR) using gene-specific primers that span the entire coding region.

The TAS2R41 cDNA (SEQ ID NO:1) was subcloned into an expression vector based on the pcDNA3.1Zeo plasmid (Invitrogen, Carlsbad, Calif., US). Within multiple cloning sites this vector contains the nucleotide sequence coding for the first 45 amino acids of the rat somatostatin receptor subtype 3 (included in SEQ ID NO:9, SST tag) to facilitate cell surface targeting of the transgene, and the nucleotide sequence coding for the herpes simplex virus (HSV) glycoprotein D epitope (HSV epitope) for facilitating immunocytochemical detection, which is included in SEQ ID NO:11, HSV Tag.

The resulting receptor cDNA in the expression vector comprises the nucleic acid sequence of TAS2R41 (SEQ ID No. 1) preceded by an SST tag (SEQ ID NO:9) and an EcoR1 site, and followed by an HSV tag (SEQ ID NO:11) in the aminoterminal to carboxyterminal direction.

The construct transfected into an expression vector is called pcDNA3.1Zeo-TAS2R41 and allows for expression of TAS2R41 amino acid sequence (SEQ ID No. 2).

### Example 2

### Transient Transfection of TAS2R41 in HEK293T/Gα16-Gustducin 44 Cells

HEK293T/G16gust44 cells were used; they were formed as described in WO 2004/055048. The host cell line HEK-293T is commercially available from the American Tissue Culture Collection (ATCC), ATCC®#CRL-11268.

On day 0, the HEK293T/G16gust44 cells were plated in 96-well black wall, clear-bottom plates at a density of 15,000 cells per well and grown overnight in growth media (Dulbecco's modified Eagle's medium (DMEM) with 10% (v/v) heat-inactivated fetal bovine serum, 2 mM L-glutamine, 100 units/ml penicillin, 100 μg/ml streptomycin).

On day 1, the media was changed to an antibiotic-free and serum-free DMEM, and the cells were transfected with Lipofectamine 2000 (Invitorgen) according to the manufacturer's recommendations.

Per well of a 96-well plate, 150 ng of vector DNA (TAS2R41 expression vectors from example 1) was diluted in 12.5 μl of DMEM. In a second tube, 0.3 μl of Lipofectamine 2000 was diluted in 12.5 μl of DMEM and incubated for 5 min at room temperature. After the 5 min, both solutions were mixed and incubated for 20 min at RT.

The growth medium in the plate was exchanged with 50 μl of DMEM and 25 μl of the lipofectamine/DNA mixture (formed in the step above) and the cells were incubated for a further 3-4 hours at 37° in a humidified atmosphere. This mixture was then replaced with an antibiotic-free, serum-containing DMEM.

The above procedure was also carried out for HEK293T/G16gust44 cells formed as described in WO 2004/055048 with the exception that no DNA was added during the process (these cells are termed Sham transfected cells);

The above procedures set out in examples 1 and 2 were also carried out using the nucleotide sequence of; SEQ ID NO: 3 allowing for expression of TAS2R41SEQ ID No. 4, SEQ ID NO: 5 allowing for expression of TAS2R41SEQ ID No. 6 and, SEQ ID NO: 7 allowing for expression of TAS2R41SEQ ID No. 8.

24 hours post transfection, the cells formed in example 2 were used in example 3.

### Example 3

### Fluo-4 Calcium Assay to Measure Activation of TAS2R41 by Cyclamate in Transiently Transfected Cells

The intracellular calcium response following addition of cyclamate was determined in HK293T cell lines transiently expressing TAS2R41 formed as described in example 2.

Each sample (receptors as well as controls) contained a final concentration of 0.02% Dimethyl sulphoxide (DMSO) to allow for comparability of all examples below.

Fluo-4AM (Invitrogen, Carlsbad, Calif., US) is a fluorescent indicator of intracellular calcium dynamics (changes in concentration) and enables the monitoring of changes in the calcium concentration, particularly an increase, in response to receptor activation occurring after agonist exposure.

On day 0, the HEK293T cells formed as described in example 2, were seeded in antibiotic-free growth medium (standard DMEM with 10% (v/v) heat-inactivated fetal bovine serum, 2 mM L-glutamine standard DMEM with 10% (v/v) heat-inactivated fetal bovine serum, 2 mM L-glutamine, 100 units/ml penicillin, and 100 μg/ml streptomycin) into black wall/clear bottom 96-well plates, coated with poly(ethylenimine) (0.005% v/v) at a concentration of 15,000 cells per well and incubated for 48 hours in humidified atmosphere (37° C., 5% CO₂).

Prior to performing the assay, the growth medium was discarded and the cells were left in a humidified atmosphere (37° C., 5% CO₂) for 1 hour with 50 μl of loading buffer consisting of 1.5 μM Fluo-4 AM and 2.5 μM probenicid (Sigma-Aldrich, St. Louis, Mo., US) in DMEM.

Following this the 96-well plate was washed 5 times with 100 μl of assay buffer (130 mM NaCl, 5 mM KCl, 10 mM 4-(2-hydroxyethyl)-1-piperazineethanesulfonic acid (HEPES), 2 mM CaCl₂, and 5 mM dextrose, pH 7.4) per well, using an automated plate washer (BioTek).

The plate was then further incubated for 30 minutes at room temperature in the dark to allow for complete de-esterification of the Fluo-4. Afterwards the plate was washed 5 times with 100 μl of assay buffer per well, and reconstituted with 100 μl of assay buffer per well.

Cyclamate solutions ranging in concentration from 250 mM to 800 mM were prepared in assay buffer.

To test receptor activation 20 μl of one of these cyclamate test solutions was added to the assay buffer of at least one well of the 96 well plate. This step was repeated until all cyclamate solutions of differing concentrations had been added to at least one well. Care was taken to ensure that only one cyclamate solution was added per well. The resulting cyclamate concentrations in the well plates ranged in concentration from 25 mM to 80 mM (This is due to the dilution of the cyclamate solution in the assay buffer present in the well).

For assay reading, the plate was placed in a Fluorometric Imaging Plate Reader (FLIPR) (FLIPR-TETRA™, Molecular Devices, Sunnyvale, Calif., US).

For each well of the plate fluorescence was continuously monitored for 20 seconds to give a signal baseline (averaged to give F₀) prior to cyclamate addition and for 120 seconds after cyclamate addition. The change in signal divided by F₀ gives ΔF/F₀ indicated in the table, with ΔF being the maximum signal occurring within the 120 seconds minus the minimum signal (occurring within the 120 seconds after cyclamate addition.

The above procedure was also independently carried out using the cells, formed as described in example 2, transfected with the TAS2R41 functional equivalent nucleotide sequences of; SEQ ID NO: 3 allowing for expression of TAS2R41SEQ ID No. 4, SEQ ID NO: 5 allowing for expression of TAS2R41SEQ ID No. 6 and, SEQ ID NO: 7 allowing for expression of TAS2R41SEQ ID No. 8.

As a control the above procedure was also carried out with the sham transfected cells formed as described in example 2.

The results are shown in the table 1.

All data was collected from at least two independent experiments each carried out in triplicate.

For the transfected cells the obtained calcium signals were corrected for the response of cells transfected with only the G Protein (G16gust44) and normalized to the fluorescence of cells prior to the stimulus using ΔF/F0 (Fmax−Fmin/F0).

| | Cyclamate Average ΔF/F0 | | | | |
|---|---|---|---|---|---|
| | T2R41 | T2R41 | T2R41 | T2R41 | |
| Concentraction of | (Nucleotide Seq | (Nucleotide Seq | (Nucleotide | (Nucleotide Seq | Sham |
| Cyclamate in mM | ID No 5)-A | ID No 7)-B | Seq ID No 1)--1 | ID No 3-3 | Transfected |
| 80 | 0.900588 | 0.895294 | 0.903529 | 0.940588 | 0.146471 |
| 75 | 0.641765 | 0.656471 | 0.701765 | 0.505294 | 0.132941 |
| 70 | 0.471176 | 0.531176 | 0.536471 | 0.373529 | 0.152941 |
| 65 | 0.396471 | 0.303529 | 0.365294 | 0.277647 | 0.138235 |
| 60 | 0.214118 | 0.185882 | 0.263529 | 0.172353 | 0.145294 |
| 55 | 0.195294 | 0.170588 | 0.144706 | 0.175882 | 0.168235 |
| 50 | 0.13 | 0.098824 | 0.141765 | 0.127647 | 0.167647 |
| 45 | 0.094706 | 0.117059 | 0.120588 | 0.156471 | 0.176471 |
| 40 | 0.103529 | 0.101176 | 0.112941 | 0.128235 | 0.171765 |
| 35 | 0.107647 | 0.111765 | 0.105882 | 0.088235 | 0.167647 |
| 30 | 0.122941 | 0.112941 | 0.102353 | 0.108824 | 0.166471 |
| 25 | 0.127647 | 0.127059 | 0.117647 | 0.118235 | 0.158824 |

It can be inferred from the results that cyclamate activates the TAS2R41 receptor and/or functional equivalents thereof at concentrations of 60 mM and higher.

### Example 4

### Identification of Modulators of the Response of TAS2R41 to Cyclamate

The change in the intracellular calcium response of TAS2R41 to cyclamate may be determined, in HK293T cell lines transiently expressing TAS2R41 formed as described in example 2, by carrying out the following method:

HEK293T cells formed as described in example 2, should be seeded in antibiotic-free growth medium (standard DMEM with 10% (v/v) heat-inactivated fetal bovine serum, 2 mM L-glutamine standard DMEM with 10% (v/v) heat-inactivated fetal bovine serum, 2 mM L-glutamine, 100 units/ml penicillin, and 100 μg/ml streptomycin) into black wall/clear bottom 96-well plates, coated with poly(ethylenimine) (0.005% v/v) at a concentration of 15,000 cells per well and incubated for 48 hours in humidified atmosphere (37° C., 5% CO₂).

Prior to performing the assay, the growth medium should be discarded and the cells left in a humidified atmosphere (37° C., 5% CO₂) for 1 hour with 50 μl of loading buffer consisting of 1.5 μM Fluo-4 AM and 2.5 μM probenicid (Sigma-Aldrich, St. Louis, Mo., US) in DMEM.

Fluo-4AM (Invitrogen, Carlsbad, Calif., US) is a fluorescent indicator of intracellular calcium dynamics (changes in concentration) and enables the monitoring of changes in the calcium concentration, particularly an increase, in response to receptor activation occurring after modulator exposure.

Following this the 96-well plate should be washed 5 times with 100 μl of assay buffer (130 mM NaCl, 5 mM KCl, 10 mM 4-(2-hydroxyethyl)-1-piperazineethanesulfonic acid (HEPES), 2 mM CaCl₂, and 5 mM dextrose, pH 7.4) per well, using an automated plate washer (BioTek).

The plate should then be incubated for a further 30 minutes at room temperature in the dark to allow for complete de-esterification of the Fluo-4. Afterwards the plate should be washed 5 times with 100 μl of assay buffer per well, and reconstituted with 100 μl of assay buffer per well.

A cyclamate solution having a concentration within the range of 600 mM to 800 mM should be prepared in assay buffer.

Following this 10 mg/L of the compound that is to be tested as a modulator (hereinafter Compound A) should be dissolved in dimethyl sulphoxide (hereinafter DSMO), this solution should then be further diluted with the previously prepared solution of cyclamate and assay buffer. The final solution should be a 250 μm solution of compound A.

For each well of the plate fluorescence should be continuously monitored for 20 seconds to give a signal baseline (averaged to give F₀).

20 μl of the prepared cyclamate solution should then be added to the assay buffer of at least two wells of the 96 well plate. The prepared compound A and cyclamate solution should then be added to at least one of the wells of the 96 well plate to which 20 μl of cyclamate solution has not already been added.

As controls, the same amount of DSMO as added to this/these well(s) in combination with compound A, should be added to at least one of the wells of the 96 well plate to which 20 μl of cyclamate solution has already been added, and to at least one of the wells of the 96 well plate to which 20 μl of cyclamate solution has not been added.

The controls will exclude any potential effect of the DSMO.

For each well of the plate fluorescence should then be continuously monitored for 120 seconds after test compound addition.

The difference in signal (ΔF) measured for those wells containing only cyclamate, those containing cyclamate and compound A, cyclamate and DSMO, and just DSMO, may then be calculated.

The difference in the signal (ΔF) measured for those wells containing only cyclamate and those containing cyclamate and compound A, should indicate whether compound A is a modulator or not. It will also indicate what type of modulator e.g. a positive change indicates an agonist or enhancer, a negative change indicates an antagonist.

The above procedure may also be independently carried out using the cells, formed as described in example 2, transfected with the TAS2R41 functional equivalent nucleotide sequences of; SEQ ID NO: 3 allowing for expression of TAS2R41SEQ ID No. 4, SEQ ID NO: 5 allowing for expression of TAS2R41SEQ ID No. 6 and, SEQ ID NO: 7 allowing for expression of TAS2R41SEQ ID No. 8.

While the receptors, nucleic acids, amino acids, expression vectors, host cells, methods and kit have been described above in connection with certain illustrative embodiments, it is to be understood that other similar embodiments may be used or modifications and additions may be made to the described embodiments for performing the same function(s). Further, all embodiments disclosed are not necessarily in the alternative, as various embodiments may be combined to provide the desired characteristics. Variations can be made by one having ordinary skill in the art without departing from the spirit and scope of the disclosure. Therefore, the receptors, nucleic acids, polypeptides, expression vectors, host cells, methods and kit should not be limited to any single embodiment, but rather construed in breadth and scope in accordance with the recitation of the attached claims.

1. A method, for identifying compounds that modulate the response of TAS2R41 to cyclamate and/or structurally related compounds comprising:
I. contacting at least one cell, or membrane thereof, expressing the nucleic acid sequence encoding TAS2R41 or a functional equivalent thereof, with cyclamate and/or a structurally related compound, and at least one test compound, and
II. measuring the effect of the test compound(s) on the response of TAS2R41 to cyclamate and/or structurally related compounds.

2. The method according to claim 1 wherein the method comprises an in vitro method.

3. The method according to claim 1 wherein the cells to be contacted with at least one test compound and cyclamate additionally comprise a G-protein.

4. The method according to claim 3 wherein the G-protein comprises the chimeric G-protein Gα16-gustducin 44.

5. The method according to claim 1 wherein the effect of the at least one test compound on the response of TAS2R41 to cyclamate and/or structurally related compounds is determined by measuring the change in concentration of the intracellular messengers IP3 and/or Ca²⁺.

6. The method according to claim 1 wherein the cells are selected from the group consisting of bacteria cells, mammalian cells, yeast cells, insect cells, amphibian cells, and worm cells.

7. The method according to claim 6 wherein the cells comprise mammalian cells.

8. The method according to claim 7 wherein the cells are selected from the group consisting of COS cells, CHO cells, HeLa cells, HEK293 cells, HEK293T cells, and HEK293 cells.

9. A kit for identifying compounds that modulate the response of TAS2R41 to cyclamate and/or structurally related compounds, comprising:
I. At least one recombinant cell expressing the nucleotide sequence encoding TAS2R41, or a functional equivalent thereof, and
II. Cyclamate and/or a structurally related compound.

10. A kit according to claim 9 wherein the cells to be contacted with at least one test compound and cyclamate additionally comprises a G-protein.

11. The kit according to claim 9 wherein the G-protein comprise the chimeric G-protein Gα16-gustducin 44.

12. The kit according to claim 9 wherein the cells are selected from the group consisting of:
bacteria cells, mammalian cells, yeast cells, insect cells, amphibian cells, and worm cells.

13. The kit according to claim 9 wherein the cells comprise mammalian cells.

14. The kit according to claim 9 wherein the cells are selected from the group consisting of: COS cells, CHO cells, HeLa cells, HEK293 cells, HEK293T cells, and HEK293 cells.

15. A method of using the kit of claim 9 for identifying compounds that modulate the response of TAS2R41 to cyclamate and/or structurally related compounds, comprising:
I. growing at least one recombinant cell expressing the nucleotide sequence encoding TAS2R41, or a functional equivalent thereof, on a solid support in a suitable culture medium,
II. adding one or more test compounds and cyclamate and/or structurally related compounds to the culture medium, and
III. measuring the effect of the one or more test compound on the response of TAS2R41 to cyclamate and/or structurally related compounds.


SEQUENCE LISTING (12 sequences)

SEQ ID NO: 1 - DNA, 924 bp, Homo sapiens
  CDS (1)..(924)
atgcaagcagcactgacggccttcttcgtgttgctctttagcctgctgagtcttctggggattgcagcgaatggcttcattgtgctggtgctgggcagggagtggctgcgatatggcaggttgctgcccttggatatgatcctcattagcttgggtgcctcccgcttctgcctgcagttggttgggacagtgcacaacttctactactctgcccagaaggtcgagtactctgggggtctcggccgacagttcttccatctacactggcacttcctgaactcagccaccttctggttttgcagctggctcagtgtcctgttctgtgtgaagattgctaacatcacacactccaccttcctgtggctgaagtggaggttcccagggtgggtgccctggctcctgttgggctctgtcctgatctccttcatcataaccctgctgtttttttgggtgaactaccctgtatatcaagaatttttaattagaaaattttctgggaacatgacctacaagtggaatacaaggatagaaacatactatttcccatccctgaaactggtcatctggtcaattcctttttctgtttttctggtctcaattatgctgttaattaattctctgaggaggcatactcagagaatgcagcacaacgggcacagcctgcaggaccccagcgcccaggctcacaccagagctctgaagtccctcatctccttcctcattctttatgctctgtcctttctgtccctgatcattgatgccgcaaaatttatctccatgcagaacgacttttactggccatggcaaattgcagtctacctgtgcatatctgtccatcccttcatcctcatcttcagcaacctcaagcttcgaagcgtgttctcgcagctcctgttgttggcaaggggcttctgggtggcctga

SEQ ID NO: 2 - protein, 307 aa, Homo sapiens
MQAALTAFFVLLFSLLSLLGIAANGFIVLVLGREWLRYGRLLPLDMILISLGASRFCLQLVGTVHNFYYSAQKVEYSGGLGRQFFHLHWHFLNSATFWFCSWLSVLFCVKIANITHSTFLWLKWRFPGWVPWLLLGSVLISFIITLLFFWVNYPVYQEFLIRKFSGNMTYKWNTRIETYYFPSLKLVIWSIPFSVFLVSIMLLINSLRRHTQRMQHNGHSLQDPSAQAHTRALKSLISFLILYALSFLSLIIDAAKFISMQNDFYWPWQIAVYLCISVHPFILIFSNLKLRSVFSQLLLLARGFWVA

SEQ ID NO: 3 - DNA, 924 bp, Homo sapiens
  CDS (1)..(924)
atgcaagcagcactgacggccttcttcgtgttgctctttagcctgctgagtcttctggggattgcagcgaatggcttcattgtgctggtgctgggcagggagtggctgcgatatggcaggttgctgcccttggatatgatcctcattagcttgggtgcctcccgcttctgcctgcagttggttgggacggtgcacaacttctactactctgcccagaaggtcgagtactctgggggtctcggccgacagttcttccatctacactggcacttcctgaactcagccaccttctggttttgcagctggctcagtgtcctgttctgtgtgaagattgctaacatcacacactccaccttcctgtggctgaagtggaggttcccagggtgggtgccctggctcctgttgggctctgtcctgatctccttcatcataaccctgctgtttttttgggtgaactaccctgtatatcaagaatttttaattagaaaattttctgggaacatgacctacaagtggaatacaaggatagaaacatactatttcccatccctgaaactggtcatctggtcaattcctttttctgtttttctggtctcaattatgctgttaattaattctctgaggaggcatactcagagaatgcagcacaacgggcacagcctgcaggaccccagcgcccaggctcacaccagagctctgaagtccctcatctccttcctcattctttatgctctgtcctttctgtccctgatcattgatgccgcaaaatttatctccatgcagaacgacttttactggccatggcaaattgcagtctacctgtgcatatctgtccatcccttcatcctcatcttcagcaacctcaagcttcgaagcgtgttctcgcagctcctgttgttggcaaggggcttctgggtggcctga

SEQ ID NO: 4 - protein, 307 aa, Homo sapiens; identical to SEQ ID NO: 2

SEQ ID NO: 5 - DNA, 924 bp, Homo sapiens
  CDS (1)..(924)
atgcaagcagcactgacggccttcttcgtgttgctctttagcctgctgagtcttctggggattgcagcgaatggcttcattgtgctggtgctgggcagggagtggctgcgatatggcaggttgctgcccttggatatgatcctcattagcttgggtgcctcccgcttctgcctgcagttggttgggacggtgcacaacttctactactctgcccagaaggtcgagtactctgggggtctcggccgacagttcttccatctacactggcacttcctgaactcagccaccttctggttttgcagctggctcagtgtcctgttctgtgtgaagattgctaacatcacacactccaccttcctgtggctgaagtggaggttcccagggtgggtgccctggctcctgttgggctctgtcctgatctccttcatcataaccctgctgtttttttgggtgaactaccctgtatatcaagaatttttaattagaaaattttctgggaacatgacctacaagtggaatacaaggatagaaacatactatttcccatccctgaaactggtcatctggtcaattcctttttctgtttttctggtctcaattatgctgttaattaattctctgaggaggcatactcagagaatgcagcacaacgggcacagcctgcaggaccccagcacccaggctcacaccagagctctgaagtccctcatctccttcctcattctttatgctctgtcctttctgtccctgatcattgatgccgcaaaatttatctccatgcagaacgacttttactggccatggcaaattgcagtctacctgtgcatatctgtccatcccttcatcctcatcttcagcaacctcaagcttcgaagcgtgttctcgcagctcctgttgttggcaaggggcttctgggtggcctga

SEQ ID NO: 6 - protein, 307 aa, Homo sapiens
MQAALTAFFVLLFSLLSLLGIAANGFIVLVLGREWLRYGRLLPLDMILISLGASRFCLQLVGTVHNFYYSAQKVEYSGGLGRQFFHLHWHFLNSATFWFCSWLSVLFCVKIANITHSTFLWLKWRFPGWVPWLLLGSVLISFIITLLFFWVNYPVYQEFLIRKFSGNMTYKWNTRIETYYFPSLKLVIWSIPFSVFLVSIMLLINSLRRHTQRMQHNGHSLQDPSTQAHTRALKSLISFLILYALSFLSLIIDAAKFISMQNDFYWPWQIAVYLCISVHPFILIFSNLKLRSVFSQLLLLARGFWVA

SEQ ID NO: 7 - DNA, 924 bp, Homo sapiens
  CDS (1)..(924)
atgcaagcagcactgacggccttcttcgtgttgctctttagcctgctgagtcttctggggattgcagcgaatggcttcattgtgctggtgctgggcagggagtggctgcgatatggcaggttgctgcccttggatatgatcctcattagcttgggtgcctcccgcttctgcctgcagttggttgggacagtgcacaacttctactactctgcccagaaggtcgagtactctgggggtctcggccgacagttcttccatctacactggcacttcctgaactcagccaccttctggttttgcagctggctcagtgtcctgttctgtgtgaagattgctaacatcacacactccaccttcctgtggctgaagtggaggttcctagggtgggtgccctggctcctgttgggctctgtcctgatctccttcatcataaccctgctgtttttttgggtgaactaccctgtatatcaagaatttttaattagaaaattttctgggaacatgacctacaagtggaatacaaggatagaaacatactatttcccatccctgaaactggtcatctggtcaattcctttttctgtttttctggtctcaattatgctgttaattaattctctgaggaggcatactcagagaatgcagcacaacgggcacagcctgcaggaccccagcacccaggctcacaccagagctctgaagtccctcatctccttcctcattctttatgctctgtcctttctgtccctgatcattgatgccgcaaaatttatctccatgcagaacgacttttactggccatggcaaattgcagtctacctgtgcatatctgtccatcccttcatcctcatcttcagcaacctcaagcttcgaagcgtgttctcgcagctcctgttgttggcaaggggcttctgggtggcctga

SEQ ID NO: 8 - protein, 307 aa, Homo sapiens
MQAALTAFFVLLFSLLSLLGIAANGFIVLVLGREWLRYGRLLPLDMILISLGASRFCLQLVGTVHNFYYSAQKVEYSGGLGRQFFHLHWHFLNSATFWFCSWLSVLFCVKIANITHSTFLWLKWRFLGWVPWLLLGSVLISFIITLLFFWVNYPVYQEFLIRKFSGNMTYKWNTRIETYYFPSLKLVIWSIPFSVFLVSIMLLINSLRRHTQRMQHNGHSLQDPSTQAHTRALKSLISFLILYALSFLSLIIDAAKFISMQNDFYWPWQIAVYLCISVHPFILIFSNLKLRSVFSQLLLLARGFWVA

SEQ ID NO: 9 - DNA, 141 bp, Rattus rattus
  CDS (1)..(141)
gccaccatggccgctgttacctatccttcatccgtgcctacgaccttggaccctgggaatgcatcctcagcctggcccctggacacgtccctggggaatgcatctgctggcactagcctggcaggactggctgtcagtggc

SEQ ID NO: 10 - protein, 47 aa, Rattus rattus
ATMAAVTYPSSVPTTLDPGNASSAWPLDTSLGNASAGTSLAGLAVSG

SEQ ID NO: 11 - DNA, 36 bp, herpes simplex virus
  CDS (1)..(36)
cagcctgaactcgctcctgaagacccggaagattaa

SEQ ID NO: 12 - protein, 11 aa, herpes simplex virus
QPELAPEDPED